(19) United States
(12) Patent Application Publication
(10) Pub. No.: US 2025/0017973 A1
(43) Pub. Date: Jan. 16, 2025

(54) Nutritional composition for cancer

(71) Applicant: N.V. Nutricia, Zoetermeer (NL)

(72) Inventors: Bram Jeroen van Bergenhenegouwen, Utrecht (NL); Bram Dorresteijn, Utrecht (NL); Hannah Rose Wardill, Adelaide (AU)

(73) Assignee: N.V. Nutricia, Zoetermeer (NL)

(21) Appl. No.: 18/882,804

(22) Filed: Sep. 12, 2024

Related U.S. Application Data
(63) Continuation of application No. PCT/EP2023/056934, filed on Mar. 17, 2023.

(30) Foreign Application Priority Data
Mar. 18, 2022 (EP) 22163105.4

(51) Int. Cl. A61K 35/20; A61K 31/23; A61P 35/00

(52) U.S. Cl. CPC A61K 35/20; A61K 31/23; A61P 35/00

(57) ABSTRACT

Provided herein is a nutritional composition having therapeutically effective amounts of a combination of (a) hydrolysed whey protein and (b) MCT for use in (i) reducing tumor growth and/or (ii) for use in improving treatment efficacy in a subject having cancer and undergoing cancer treatment.

### FIELD OF THE INVENTION

The present invention is in the field of nutritional compositions for reducing tumor growth or improving cancer treatment efficacy.

### BACKGROUND ART

Cancer treatments have improved greatly throughout the past century with the development of various therapies including surgery, radiation, hormone therapy, chemotherapy, and immunotherapy. Chemotherapy remains one of the most common treatments for cancer. New chemotherapy drugs and combination chemotherapy drug regimens are constantly being developed and tested to increase potency and reduce side-effects. The improvement of treatment of cancer has still high significance as the number of people suffering from neoplastic diseases will further increase in the coming years, particularly due to the aging populations. Even if survival rates have improved dramatically over the past few decades in particular in children and younger patients, there is still an essential need in improving efficacy of the treatment of cancer patients.

Chemotherapy in the simplest sense is the treatment of an ailment by chemicals comprising neoplastic diseases, i.e., benign or malignant tumors. All chemotherapeutic agents cause more or less severe side effects, which make it sometimes even difficult to treat a patient dependent on the general health condition of the patient. There exists a high interest in both increasing the efficacy of chemotherapeutic agents and decreasing the side effects at the same time to improve the treatment of neoplastic diseases.

There remains a need in the art to provide nutritional compositions providing beneficial effects to subjects undergoing cancer treatment.

### SUMMARY OF THE INVENTION

The inventors have observed that administration of therapeutically effective amounts of a combination of (a) hydrolysed whey protein and (b) medium chain triglycerides (MCT) to a subject having cancer and undergoing cancer treatment beneficially reduces tumor growth and improves cancer treatment efficacy. The reduction in tumor growth is a reduction compared to the tumor growth of the subject undergoing cancer treatment not receiving therapeutically effective amounts of the combination of the invention. Also the improvement in treatment efficacy is an improvement compared to treatment efficacy of the subject undergoing cancer treatment not receiving therapeutically effective amounts of the aforementioned combination. The combination is preferably provided in the form of a nutritional composition.

The nutritional composition according to the invention can therefore beneficially be used in the treatment of cancer, wherein the nutritional composition is administered enterally, preferably orally to a subject undergoing cancer treatment.

The invention thus pertains to a nutritional composition comprising therapeutically effective amounts of a combination of (a) hydrolysed whey protein and (b) MCT for use in (i) reducing tumor growth and/or (ii) improving treatment efficacy in a subject having cancer and undergoing cancer treatment, and to therapeutically effective amounts of a combination of (a) hydrolysed whey protein and (b) MCT for use in (i) reducing tumor growth and/or (ii) improving treatment efficacy in a subject having cancer and undergoing cancer treatment.

Reducing tumor growth and/or improving treatment efficacy involves any of reducing cancer cell mitosis, increasing cancer cell apoptosis, improving cancer cell sensitivity to cancer treatment and/or reducing the amount and/or occurrence of metastasis.

The composition of the present invention may be provided along with cancer treatment selected from surgical treatment, chemotherapy, radiation therapy, immunotherapy, or combinations thereof.

Worded differently, the invention pertains to a method for i) reducing tumor growth and/or (ii) improving treatment efficacy in subjects having cancer, preferably in a subject having cancer and undergoing cancer treatment comprising administering to the subject a nutrition composition comprising therapeutically effective amounts of (a) hydrolysed whey protein and (b) MCT. Also, the invention pertains to the use of therapeutically effective amounts of a combination or nutritional composition as mentioned here above, in the manufacture of a product for i) and/or ii).

In particular, the present invention provides nutritional compositions, method, use and the nutritional composition for use in i) reducing tumor growth and/or (ii) improving treatment efficacy in subjects having cancer and undergoing cancer treatment.

The invention further pertains to a nutritional composition for patients undergoing cancer treatment essentially consisting of a) hydrolysed whey protein and (b) MCT and wherein the hydrolysed whey protein comprises more than 50 wt. % of whey peptide fragments having a molecular weight of between 0.5 and 3 kDa.

### BRIEF DESCRIPTION OF FIGURES

The present invention will be discussed in more detail below, with reference to the attached drawings.

Four groups of rats were tumor bearing and exposed to a treatment regimen followed by assessment of the tumor growth: Vehicle and Test diet, methotrexate (MTX) and Test diet, vehicle and Control diet, MTX and Control diet:

FIG. 1A The number of animals with detectable (i.e. identifiable, dissected and weighted) tumors at necropsy in 4 treatment groups. The number of animals with tumors still present after methotrexate (MTX) treatment was significantly higher in the group fed the Control diet (5/8) compared to the group fed the Test diet (0/8). Chi-squared test p<0.0001.

FIG. 1B. Tumour burden at necropsy in all 4 treatment groups expressed as relative burden of tumor size in cm3 (v/w) as percentage of the body weight of the animal. Data shown as mean±SEM (n=8 per group), analysed with one-way ANOVA with Tukey's post-hoc test.

FIG. 1C. Tumour burden at necropsy in all 4 treatment groups expressed as relative burden of tumor size in grams (w/w) as percentage of the body weight of the animal. Data shown as mean±SEM (n=8 per group), analysed with one-way ANOVA with Tukey's post-hoc test.

### LIST OF EMBODIMENTS

1. A nutritional composition comprising therapeutically effective amounts of a combination of (a) hydrolysed whey protein and (b) MCT for use in (i) reducing tumor growth and/or (ii) for use in improving treatment efficacy in a subject having cancer and undergoing cancer treatment.
2. The nutritional composition for use according to embodiment 1 wherein the hydrolysed whey protein comprises more than 50 wt. % of whey peptide fragments having a molecular weight of between 0.5 and 3 kDa based on total weight of the hydrolysed whey protein.
3. The nutritional composition for use according to embodiments 1 and 2, wherein the hydrolysed whey protein comprises more than 130 wt. %, preferably more than 135 wt. % of whey proteins having a molecular weight below 7500 Da based on total weight of the hydrolysed whey protein.
4. The nutritional composition for use according to the preceding embodiments wherein the hydrolysed whey protein comprises less than 5 wt %, preferably less than 4 wt %, more preferably less than 2 wt %, most preferably less than 1.5 wt % of whey protein fragments having a molecular weight of 5 kDa or more based on total weight of the hydrolysed whey protein.
5. The nutritional composition for use according to the preceding embodiments wherein the nutritional composition comprises (I) a protein fraction comprising at least 80 wt % hydrolysed whey protein based on the total weight of the protein fraction and (II) a lipid fraction comprising at least 40 wt % MCTs based on total weight of the lipid fraction.
6. The nutritional composition for use according to embodiment 5 wherein the (I) protein fraction comprises at least 85 wt. % hydrolysed whey protein, more preferably at least 90 wt. %, even more preferably at least 95 wt. %, and most preferably about 100 wt. % of hydrolysed whey protein based on total weight of the protein fraction.
7. The nutritional composition for use according to embodiments 5 and 6 wherein the MCTs provide 9-25% of the total energy content of the nutritional composition, more preferably 10-22% and most preferably 11-19% of the total energy content of the nutritional composition.
8. The nutritional composition for use according to embodiments 5 to 7 wherein the protein fraction provides 5 to 45%, preferably 10 to 35%, even more preferably 15 to 30% of the total energy content of the nutritional composition.
9 The nutritional composition for use according to embodiments 5 to 8 wherein the nutritional composition comprises a (III) carbohydrate fraction.
10. The nutritional composition for use according to embodiments 5 to 9 wherein the carbohydrate fraction provides 30-70% of the total energy content of the nutritional composition, more preferably 35-65% and most preferably 45-62.5% of the total energy content of the nutritional composition.
11. The nutritional composition for use according to the preceding embodiments wherein (i) reducing tumor growth and/or (ii) improving treatment efficacy in a subject having cancer and undergoing cancer treatment involves
   reducing tumor growth,
   reducing cancer cell mitosis,
   increasing cancer cell apoptosis,
   improving cancer cell sensitivity to cancer treatment and/or
   reducing the amount and/or occurrence of metastasis compared to the reduction in tumor growth and/or improvement of treatment efficacy in a subject having cancer and undergoing cancer treatment without use of the nutritional composition.
12. The nutritional composition for use according to the preceding embodiments wherein cancer treatment is selected from surgical treatment, chemotherapy, radiation therapy, immunotherapy, or combinations thereof.
13. The nutritional composition for use according to the preceding embodiments wherein the cancer treatment is at least chemotherapy.
14. The nutritional composition for use according to the preceding embodiments wherein the nutritional composition is in the form of a liquid.
15. The nutritional composition for use according to the preceding embodiments wherein the whey protein hydrolysate has a degree of hydrolysis of about 15 to 25%.

### DESCRIPTION OF THE INVENTION

It has surprisingly been found that a nutrition composition comprising therapeutically effective amounts of a combination of (a) hydrolysed whey protein and (b) MCT reduces tumor growth and/or (ii) improves treatment efficacy in subjects undergoing cancer treatment. It was found that use of the composition has a great potential in cancer treatment. A study in an animal model surprisingly revealed a delay in tumor development and a decrease in tumor size in animals receiving the nutritional composition according to the invention.

Without being bound by theory it postulated that the surprisingly beneficial effect on tumor growth by the combination of hydrolysed whey proteins and MCT may be associated with a combination of health promoting and modulatory effects of the combination of nutrients. Additionally the use of the nutritional composition according to the invention prevented or reduced weight loss during cancer treatment and thereby aided in maintaining overall health.

The invention thus pertains to a nutritional composition comprising therapeutically effective amounts of a combination of (a) hydrolysed whey protein and (b) MCT for use in (i) reducing tumor growth and/or (ii) improving treatment efficacy in subjects undergoing cancer treatment.

Worded differently, the invention pertains to a method for i) reducing tumor growth and/or (ii) improving treatment efficacy in subjects having cancer, preferably in a subject having cancer and undergoing cancer treatment comprising administering to the subject a nutritional composition comprising therapeutically effective amounts of (a) hydrolysed whey protein and (b) MCT.

The invention further pertains to the use of a nutritional composition comprising therapeutically effective amounts of a combination of (a) hydrolysed whey protein and (b) MCT in the manufacture of a product for i) reducing tumor growth and/or (ii) improving treatment efficacy in subjects having cancer.

In particular, the present invention provides nutritional compositions, methods of use and uses to i) reducing tumor growth and/or (ii) improving treatment efficacy in subjects having cancer and undergoing cancer treatment. Also, the invention pertains to the use of therapeutically effective amounts of a combination or nutritional composition as mentioned here above, in the manufacture of a product for i) and/or ii).

The invention preferably pertains to a nutritional composition for (use in) patients undergoing cancer treatment essentially consisting of a) hydrolysed whey protein and (b) MCT and wherein the hydrolysed whey protein comprises more than 50 wt. % of whey peptide fragments having a molecular weight of between 0.5 and 3 kDa. Preferably the hydrolysed whey protein further comprises more than 5 wt. %, preferably more than 10 wt. %, more preferably more than 15 wt % of peptides or proteins having a molecular weight below 375 Da based on total protein of the hydrolysed whey protein fraction. Preferably the hydrolysed whey protein further comprises more than 20 wt. %, preferably more than 25 wt. % of whey proteins having a molecular weight below 500 Da.

The present invention has been described above with reference to a number of exemplary embodiments as shown in the drawings. Modifications and alternative implementations of some parts or elements are possible, and are included in the scope of protection as defined in the appended claims.

### Definitions

Throughout this application, the following terminology and abbreviations may be used.

“Nutritional composition” as used herein means a substance or formulation that satisfies at least a portion of a subject's nutrient requirements. The terms “nutritional(s)”, “nutritional formula(s)”, “enteral nutritional(s)”, and “nutritional supplement(s)” are used as non-limiting examples of nutritional composition(s) throughout the present disclosure. Moreover, “nutritional composition(s)” may refer to liquids, powders, gels, pastes, solids, concentrates, suspensions, or ready-to-use forms of enteral formulas, oral formulas. The term nutritional composition refers to a synthetically produced food composition. Thus, nutritional compositions are artificial nutritional products obtained by mixing/dissolving bulk ingredients whereby said ingredients are typically provided in solid form (e.g. powders) or liquid from (e.g. oils, water, syrup). The term “nutritional composition” therewith excludes “food”, i.e. non-modified natural food products, such as meat, vegetables, fruits in their natural form and conventionally prepared (e.g. cooked) meals or drinks like tea, coffee or juices.

A medium-chain triglyceride (MCT) is a triglyceride in which all three fatty acid moieties are medium-chain fatty acid moieties. As defined herein, medium-chain fatty acids (MCFA) are fatty acids that have 6 to 12 carbon atoms. Medium-chain fatty acids with 8 carbon atoms may be referred to herein as “C8 fatty acids” or “C8.” Medium-chain fatty acids with 10 carbon atoms may be referred to herein as “C10 fatty acids” or “C10.”

The term “fatty acid moiety” refers to the part of the MCT that originates from a fatty acid in an esterification reaction with glycerol. In a non-limiting example, an esterification reaction between glycerol and only octanoic acid would result in a MCT with octanoic acid moieties. In another non-limiting example, an esterification reaction between glycerol and only decanoic acid would result in a MCT with decanoic acid moieties. Octanoic acid (also known as caprylic acid) is a saturated fatty acid with the formula CH(CH2)₆COOH. Decanoic acid (also known as capric acid) is a saturated fatty acid of the formula CH(CH2)₆COOH.

The term “supplement”, or “dietary supplement”, as used herein, refers to a nutritional product that provides nutrients to an individual that may otherwise not conveniently be consumed in sufficient quantities by said individual. Supplements typically provide the selected nutrients while not representing a significant portion of the overall nutritional needs of the subject. Typically they do not represent more than 0.1%, 1%, 5%, 10% of the daily energy need of the subject.

In the context of the invention a subject adhering to or taking the nutritional composition according to the invention is a mammal, preferably a human.

The term “cancer treatment” or “during cancer treatment” as used herein means any period of time between the initial cancer diagnosis or start of the first form of cancer treatment until the end of a last cancer treatment administration. For example, in a subject that undergoes several rounds of chemotherapy that are spread by several weeks wherein no chemotherapy is administered the cancer treatment is intended to mean the entire period between the start of the first dose of chemotherapy and metabolic clearance of the last dose of chemotherapy which typically lasts up to several weeks after administration of a dose of chemotherapy.

The energy provided by nutrients is calculated using the Atwater calculation factors 9 kcal per g lipid, 4 kcal per gram protein or gram digestible carbohydrate and zero kcal for the other components in the product.

The term “hydrolysed whey protein” herein refers to whey protein that has been processed and/or treated in a manner intended to break peptide bonds. The term “hydrolysed whey protein” thus refers to whey protein that is at least partially hydrolysed. Intentional hydrolysis may be carried out by e.g. treating intact protein with one or more enzymes and/or with acids or bases. Hydrolysates may be characterised as “partial” or “extensive” depending on the degree to which the hydrolysis reaction is carried out. In the context of the present invention, a partial hydrolysate is a hydrolysate in which 60 percent of the protein/peptides has a molecular weight of less than 1000 Daltons, whereas an extensive hydrolysate is one in which at least 95 percent of the protein/peptides has a molecular weight of less than 1000 Daltons.

A measure for the extent of hydrolysation of the whey protein is the “degree of hydrolysation” (DH). The degree of hydrolysation is defined as the percentage of the total number of peptide bonds in a protein that has been cleaved during hydrolysis. The degree of hydrolysis of a protein may e.g. be determined by the trinitrobenzenesulphonic acid (TNBS) procedure, as known in the art (Adler-Nissen, J. Agr. Food Chem. 1979, 27 (6), 1256). When whey protein is subjected to a hydrolysis process, the source of whey protein may already comprise a certain (small) amount of peptide fractions, before being subjected to the hydrolysis process. The values for the degree of hydrolysation as described herein are corrected for this presence of peptide fractions in the whey protein source, in other words, the values for the degree of hydrolysation are corrected for the natural degree of hydrolysation of whey protein. Herein, the degree of hydrolysation thus relates to the additional hydrolysation that was obtained via the intentional hydrolysis process.

In this document and in its claims, the verb “to comprise” and its conjugations is used in its non-limiting sense to mean that items following the word are included, but items not specifically mentioned are not excluded. “Consisting essentially of” means that the composition contains the specified active ingredients and possibly additional compounds, provided these do not materially affect the essential characteristics of the composition. In addition, reference to an element by the indefinite article “a” or “an” does not exclude the possibility that more than one of the element is present, unless the context clearly requires that there be one and only one of the elements. The indefinite article “a” or “an” thus usually means “at least one”. Where reference is made to % herein it means wt % unless indicated differently.

### Nutritional Composition

The present invention relates to a combination or nutritional composition comprising therapeutically effective amounts of a combination of (a) hydrolysed whey protein and (b) medium chain triglycerides (MCT) for use in reducing tumor growth and/or (ii) for use in improving treatment efficacy in a subject having cancer and undergoing cancer treatment.

The nutritional composition preferably is an enteral nutritional composition. The nutritional composition may be formulated as complete nutrition, and potentially serve as the sole source of nutrition. Alternatively, the nutritional composition may be in the form of a food supplement. Preferably the nutritional composition is in liquid form.

The nutritional composition preferably comprises (I) a protein fraction comprising at least 80 wt % hydrolysed whey protein based on the total weight of the protein fraction and (II) a lipid fraction comprising at least 40 wt % MCTs based on total weight of the lipid fraction.

In a preferred aspect MCTs provide 9-25% of the total energy content of the nutritional composition and the protein fraction provides 5 to 45% of the total energy content of the nutritional composition. In a further preferred aspect the nutritional composition comprises a (III) carbohydrate fraction wherein the carbohydrate fraction provides 30-70% of the total energy content of the nutritional composition, more preferably 35-65% and most preferably 45-62.5% of the total energy content of the nutritional composition.

In a preferred aspect the hydrolysed whey protein of the nutritional composition comprises more than 40 wt. % of whey peptide fragments having a molecular weight of between 0.5 and 3 kDa. Furthermore the hydrolysed whey protein preferably comprises more than 10 wt. %, preferably more than 15 wt. % of whey protein fragments having a molecular weight below 500 Da. The hydrolysed whey protein further preferably comprises more than 50 wt %, more preferably more than 60 wt %, even more preferably more than 70 wt % of whey protein fragments having a molecular weight below 1.5 kDa based on total protein of the hydrolysed whey protein fraction.

In a further preferred aspect the nutritional composition comprises a protein fraction comprising at least 80 wt. % hydrolysed whey protein based on total weight of the protein fraction. The nutritional composition further comprises a lipid fraction comprising at least 30 wt. % MCTs based on total weight of the lipid fraction. In a further aspect the nutritional composition may comprise a carbohydrate fraction, said carbohydrate fraction may comprise fibers and/or digestible carbohydrates.

Further preferred aspects and ranges of the nutritional fractions of the nutritional composition are discussed hereinafter.

### Protein Fraction

In an aspect of the invention the nutritional composition comprises therapeutically effective amounts of (a) hydrolysed whey protein in an amount effective for reducing tumor growth and/or (ii) improving treatment efficacy in a subject having cancer, in a preferred aspect a subject having cancer and undergoing cancer treatment.

The nutritional composition according to the invention preferably comprises 5-10 g of protein per 100 ml of the composition. The total protein that is present in the nutritional composition, i.e. the combination of all proteins present, may also be referred to as the “protein fraction” of the nutritional composition. The nutritional composition thus preferably comprises a protein fraction of 5-10 g per 100 ml of the composition. In a preferred aspect the nutritional composition comprises a protein fraction of 6-9 g per 100 ml of the composition, more preferably of 6.5-8.5 g. Most preferably the composition comprises a protein fraction of 7-8 g per 100 ml of the composition.

According to an embodiment of the present invention, the protein fraction provides 5 to 45%, preferably 10 to 35%, even more preferably 15 to 30% of the total energy content of the nutritional composition. Most preferably the protein fraction of the composition provides 18 to 25% of the total energy of the nutritional composition.

At least 80 wt. % of the protein fraction in the nutritional composition is hydrolysed whey protein, preferably at least 85 wt. % of the protein is hydrolysed whey protein, more preferably at least 90 wt. %, even more preferably at least 95 wt. %, and most preferably about 100 wt. % of the protein fraction is hydrolysed whey protein. In other words, the protein fraction of the nutritional composition comprises 80 wt. % or more, based on the total weight of the protein fraction, of hydrolysed whey protein.

The nutritional composition according to the invention thus essentially comprises hydrolysed whey protein. The whey protein hydrolysate is preferably provided by whey protein hydrolysate derived from mammalian milk, preferably milk from a species of the genus Bos, Bison, Bubalus or Capra, more preferably from genus Bos, most preferably from cow's milk (Bos taurus).

A suitable source of whey protein in the nutritional composition is a mixture of acid whey protein and demineralised sweet whey protein. Acid whey and sweet whey are commercially available. Sweet whey is the by-product of rennet-coagulated cheese and comprises caseinoglycomacropeptide (CGMP), and acid whey (also called sour whey) is the by-product of acid-coagulated cheese, and does not contain CGMP. Suitable sources for the whey protein hydrolysate are demineralised whey (Deminal, Friesland Campina, the Netherlands) and/or whey protein concentrate (WPC80, Friesland Campina, the Netherlands). The whey protein preferably comprises acid whey, more preferably at least 50 wt %, more preferably at least 70 wt % acid whey, based on total whey protein. Acid whey has an improved amino acid profile compared to sweet whey protein. Hydrolysis may be achieved using a mixture of microbial endopeptidases and exopeptidases using the method as described in example 1 of WO 2011/151059, herein incorporated by reference.

The hydrolysed whey preferably comprises less than 5 wt %, preferably less than 4 wt %, more preferably less than 2 wt %, most preferably less than 1.5 wt % of peptides or proteins with a size of 5 kDa or above, based on total protein of the hydrolysed whey protein fraction. It is preferred that more than 15 wt % of whey peptides or proteins present in the hydrolysed whey protein fraction has a size of 1 kDa or above, based on total protein of the hydrolysed whey protein fraction, more preferably at least 20 wt %, more preferably at least 25 wt %, based on total protein of the hydrolysed whey protein fraction. It is further preferred that more than 2 wt % of peptides or proteins present in the hydrolysed whey protein fraction has a size of 3 kDa or above, more preferably at least 4 wt %, more preferably at least 6 wt %, based on total protein of the hydrolysed whey protein fraction.

Preferably more than 40 wt % of whey peptides or proteins present in the hydrolysed whey protein fraction has a size between 0.5 and 3 kDa, more preferably more than 45 wt %, more preferably more than 50 wt %, based on total protein of the hydrolysed whey protein fraction. It is preferred that more than 30 wt % of whey peptides or proteins present in the hydrolysed whey protein fraction has a size between 0.5 and 1 kDa, more preferably more than 35 wt %, more preferably more than 40 wt %, based on total protein of the hydrolysed whey protein fraction. It is further preferred that more than 20 wt % of whey peptides or proteins present in the hydrolysed whey protein fraction has a size between 375 and 750 Da, more preferably more than 25 wt %, even more preferably more than 30 wt %, based on total protein of the hydrolysed whey protein fraction.

The hydrolysed whey protein preferably comprises preferably more than 5 wt. %, preferably more than 10 wt. %, more preferably more than 15 wt % of peptides or proteins having a molecular weight below 375 Da based on total protein of the hydrolysed whey protein fraction. The hydrolysed whey protein further preferably comprises preferably more than 30 wt. %, preferably more than 35 wt. %, preferably more than 40 wt %, even more preferably more than 45 wt % of peptides or proteins having a molecular weight below 750 Da based on total protein of the hydrolysed whey protein fraction. The hydrolysed whey protein further preferably comprises preferably more than 50 wt. %, preferably more than 60 wt. %, more preferably more than 65 wt % of peptides or proteins having a molecular weight below 1250 Da based on total protein of the hydrolysed whey protein fraction. The hydrolysed whey protein further preferably comprises more than 50 wt %, more preferably more than 60 wt %, even more preferably more than 70 wt % of whey protein fragments having a molecular weight below 1.5 kDa based on total protein of the hydrolysed whey protein fraction.

In a further embodiment it is preferred that the hydrolysed whey protein comprises 5 wt. % or less intact protein, preferably 2 wt. % or less, more preferably 1 wt. % or less, most preferably 0.5 wt % or less intact protein, based on total protein in the hydrolysed whey protein. Intact protein may be removed from hydrolysed whey protein by a filtration process, as is known in the art.

The whey protein hydrolysate of the nutritional composition may in an embodiment be characterised in that it comprises between 50 and 80 wt %, preferably between 60 and 70 wt % peptides with a molecular weight below 1 kDa, between 15 and 40 wt %, more preferably between 20 and 35 wt % peptides with a molecular weight between 1 kDa and 3 kDa, and between 0.5 and 4 wt %, more preferably between 1 and 2 wt % of peptides or proteins with a molecular weight above 5 kDa, all based on total protein in the hydrolysed whey protein fraction.

The size distribution of the peptides in the protein hydrolysate can be determined by means of size exclusion high pressure liquid chromatography as known in the art. Saint-Sauveur et al. “Immunomodulating properties of a whey protein isolate, its enzymatic digest and peptide fractions” Int. Dairy Journal (2008) vol. 18 (3) pages 260-270 describes an example thereof. In short, the total surface area of the chromatograms is integrated and separated into mass ranges expressed as percentage of the total surface area. The mass ranges are calibrated using peptides/proteins with a known molecular mass.

In a particular embodiment of the nutritional composition according to the invention, the hydrolysed whey protein has a degree of hydrolysation of 10% or more. More preferably, the degree of hydrolysation is 15% or more, even more preferably 18% or more. It is particularly preferred that the degree of hydrolysation of the whey protein is in the range of 15 to 25%, even more particularly in the range of 18 to 22%. In an embodiment the degree of hydrolysis of the hydrolysed whey protein may be about 20%. The degree of hydrolysation as used herein is corrected for the natural degree of hydrolysation of the whey protein source, i.e. the whey protein that was used for the preparation of the hydrolysed whey protein.

Processes for the manufacturing of hydrolysed whey protein are known in the art. Hydrolysed whey protein may for example be prepared by subjecting whey protein concentrate (WPC), whey protein isolate (WPI), serum protein concentrate (SPC) or serum protein isolate (SPI) to an enzymatic hydrolysis process. Alternatively, hydrolysed whey protein may be prepared by acid or base hydrolysis of whey protein. WPC, WPI, SPC and SPI may be obtained by processes known in the art, such as the processing of sweet whey or acid whey, ultrafiltration or microfiltration processes. Whey protein concentrate (WPC) typically comprises about 35 to about 80 wt. % protein, based on dry matter. Whey protein isolate (WPI) typically comprises about 85 wt. % or more protein, based on dry matter. Serum protein concentrate (SPC) typically comprises about 60 wt. % protein, based on dry matter. Serum protein isolate (SPI) typically comprises 5 about 85 wt. % protein, based on dry matter.

Hydrolysed whey protein is further also commercially available from several suppliers. Examples of suitable sources of hydrolysed whey protein include products of the Hyvital hydrolysed whey protein range of FrieslandCampina Domo and hydrolysed whey protein products available from, amongst others, Kerry (Ireland), Arla (Denmark) and Fonterra (New Zealand).

### Lipids

The nutritional composition according to the invention preferably comprises a lipid fraction, preferably a lipid fraction suitable for nutrition as known in the art.

The lipid fraction of the present composition preferably provides 2 to 5 g, more preferably 3 to 4.5 g per 100 kcal of the composition. When in liquid form, the composition preferably comprises 3 to 7.5 g lipid per 100 ml, more preferably 4 to 6 g per 100 ml.

The lipid fraction preferably provides more than 15% of the energy of the nutritional composition. Preferably the lipid fraction of the nutritional composition provides 15-45% of the total energy content of the composition, more preferably 20-40% and most preferably 25-35% of the total energy content of the nutritional composition.

The amount of lipid fraction can be determined by applying the methods known in the art for measuring fat content in the food matrix as applicable. For example, fat content for general foods is determined by applying AOAC® official method 983.23, while the Roese-Gottlieb method (AOAC® 932.06) is better applicable for products based on dried milk (Lehner, R., Estoppey, A., (1954) Mitt. Lebensmitteluntersuchung Hyg. 54:183-185). The amount of individual lipid components can be determined by applying methods specifically designed for measuring that specific component or by fractionating the fat fraction isolated from the extraction of the chloroform-niethanol fraction as given in the 983.23 method.

The lipid component typically comprises lipids that are a source of essential fatty acids alpha-linolenic acid (ALA, C-18:3) and linoleic acid (LA, C-18:2). LA and/or ALA may be provided as free fatty acids, in triglyceride form, in diglyceride form, in monoglyceride form, in phospholipid form, or as a mixture of one of more of the above. Preferably the present composition contains at least one, preferably at least two lipid sources. Exemplary lipid sources that may be used herein include soybean oil, rape seed oil (such as colza oil, low erucic acid rape seed oil and canola oil), high oleic sunflower oil, high oleic safflower oil and olive oil.

In a preferred aspect at least 20 wt. % of the lipid fraction in the nutritional composition are from a lipid source providing LA and/or ALA, preferably at least 25 wt. % of the lipid fraction in the nutritional composition, more preferably at least 30 wt. % of the lipid fraction in the nutritional composition are from a lipid source providing LA and/or ALA, based on total weight of the lipid fraction.

The LA:ALA weight ratio is preferably in the range of 7:1 to 12:1, preferably 8:1 to 12:1, more preferably 7:1 to 10:1.

### Medium Chain Triglycerides

In an aspect of the invention the nutritional composition comprises therapeutically effective amounts of (b) MCTs in an amount effective for reducing tumor growth and/or (ii) improving treatment efficacy in a subject having cancer, in a preferred aspect a subject having cancer and undergoing cancer treatment.

The nutritional composition according to the invention thus comprises MCTs. MCTs are a type of fat found in a small number of foods, such as coconut oil and palm kernel oil. MCTs are defined to be linear or branched, preferably linear, saturated carboxylic acids having six to twelve carbon atoms.

When hydrolysed, these oils provide concentrated sources of MCFAs with chain lengths of primarily 8 (caprylic or octanoic acid) and 10 (capric or decanoic acid) carbon atoms. Hence, in practice, when MCTs are administered, this is often limited to fatty acids with 8- and 10-carbon chains, although (theoretically) MCFAs also include carbon chains of 6 and 12 carbon atoms. Hence, the MCFAs according to the invention are preferably selected from MCTs originating from coconut oils and/or palm kernel oils. The chain length of the MCFAs according to the invention is 6, 7, 8, 9, 10, 11 or 12, preferably 8, 9 or 10, most preferably 8 or 10 carbon atoms long, or any mixture thereof. The amounts of MCT provided are based on the weight of MCTs present in the composition and corrected for the presence of other fatty acids in an MCT oil blend.

In a preferred embodiment the MCT fraction comprises caprylic acid (C8:0) and capric acid (C10:0), preferably at a weight ratio of between about 3.5:2 and about 2.5:2, more preferably between about 3.2:2 and about 2.8:2.

The lipid fraction of the present composition preferably comprises 1 to 3 g, more preferably 1.5 to 2.5 g of MCTs per 100 kcal of the composition. When in liquid form, the nutritional composition preferably comprises 2 to 4 g MCTs per 100 ml, more preferably 2.5 to 3.5 g MCTs per 100 ml.

The MCTs preferably provide more than 9% of the energy of the nutritional composition. Preferably the MCTs in the nutritional composition provide 9-25% of the total energy content of the nutritional composition, more preferably 10-22% and most preferably 11-19% of the total energy content of the nutritional composition.

At least 40 wt. % of the lipid fraction in the nutritional composition are MCTs, preferably at least 50 wt. % of the lipid fraction in the nutritional composition are MCTs, more preferably at least 55 wt. % lipid fraction in the nutritional composition are MCTs based on total weight of the lipid fraction.

### Carbohydrate

The nutritional composition according to the invention comprises a carbohydrate fraction, which may optionally include a source of dietary fibers. In the context of the present invention, the term “fiber” refers to one or more non-digestible oligosaccharides. Advantageously, the non-digestible oligosaccharide is water-soluble (according to the method disclosed in L. Prosky et al, J. Assoc. Anal. Chem 71:1017-1023, 1988). Non-digestible oligosaccharides are not digested in the intestine by the action of digestive enzymes present in the human upper digestive tract (small intestine and stomach) but instead are fermented by the human intestinal microbiota.

The nutritional composition according to the invention may comprise dietary fiber, which term also includes mixtures of dietary fibers. In one embodiment, when fibers are present in the nutritional composition the fermentable dietary fiber or fibers are selected from the group consisting of oligofructose, inulin, wheat bran, gum arabic, soy polysaccharides, oat fiber, galactooligosaccharides, locus bean gum, guar gum, pectin, hydrolysed pectin and mixtures thereof. In a preferred embodiment, the dietary fiber or fibers are selected from the group consisting of oligofructose, inulin, resistant starch, cellulose, methylcellulose preferably hydroxypropyl methylcellulose, wheat bran, gum arabic, soy polysaccharides, oat fiber, galactooligosaccharides, locus bean gum, guar gum, pectin, hydrolysed pectin and mixtures thereof. The terms ‘gum arabic’ and ‘arabic gum’ are used interchangeably. In one embodiment, one fiber is used, which is galactooligosaccharides. Alternatively, mixtures of distinct dietary fibers may be used, such as mixtures of at least 2, at least 3, at least 4, at least 5, at least 6 or even at least 7 distinct dietary fibers are used, preferably selected from the above list of preferred dietary fibers.

In one embodiment of the invention the dietary fiber comprises galactooligosaccharide. In another embodiment of the invention the dietary fiber comprises a mixture of galactooligosaccharide and resistant starch. In another embodiment the dietary fiber is a mixture of galactooligosaccharide, resistant starch and additional fibers.

The nutritional composition of the invention may deliver about 5 g to about 15 g of fibers per day, e.g. about 9 g per day

The nutritional composition according to the invention may comprise further carbohydrates, preferably the present composition comprises a digestible carbohydrate fraction. Typically, digestible carbohydrates that are known in the art to be suitable for use in nutritional compositions are used. Preferably, the digestible carbohydrate is selected from digestible polysaccharides (e.g. starch, maltodextrin), digestible monosaccharides (e.g. glucose, fructose, sucrose), and digestible disaccharides (e.g. lactose, sucrose). Particularly suitable is starch and/or maltodextrin. In one embodiment, the composition does not comprise lactose.

The nutritional composition preferably comprises 10 to 20 g, more preferably 12.5 to 17.5 g of digestible carbohydrates per 100 kcal of the nutritional composition. When in liquid form, the nutritional composition preferably comprises 15 to 25 g digestible carbohydrates per 100 ml, more preferably 17.5 to 22.5 g digestible carbohydrates per 100 ml.

The digestible carbohydrates preferably provide more than 30% of the energy of the nutritional composition. Preferably digestible carbohydrates of the nutritional composition provides 30-70% of the total energy content of the nutritional composition, more preferably 35-65% and most preferably 45-62.5% of the total energy content of the nutritional composition.

### Therapeutic Application

Nutritional support during treatment of cancer is widely used in cancer care. The nutritional composition of the present invention serves the purpose of being a disease modifying nutrition while at the same time providing nutritional support, and thereby ensures two important dimensions in a cancer treatment regime, all in one single composition. Such dietary treatment with the composition of the invention might be introduced into classic protocols of human cancer therapy as a new, non-toxic and easily applicable adjuvant cancer therapy without any additional risk to the patient.

Accordingly, an aspect of the present disclosure is a nutritional composition comprising therapeutically effective amounts of a combination of (a) hydrolysed whey protein and (b) MCTs, the nutritional composition comprising the combination of hydrolysed whey and MCTs in an amount effective for reducing tumor growth and/or (ii) improving treatment efficacy in a subject having cancer and undergoing cancer treatment.

Reducing tumor growth and/or improving treatment efficacy involves any of reducing cancer cell mitosis, increasing cancer cell apoptosis, improving cancer cell sensitivity to cancer treatment and/or reducing the amount and/or occurrence of metastasis.

Any type of cancer may benefit from use of the nutritional composition according to the invention. Exemplary types of cancers that may be treated with the nutritional composition of the invention include breast cancer, prostate cancer, lung cancer and skin cancer. Even further examples of types of cancers that can be treated with the composition of the invention are adrenal cancer, anal cancer, bile duct cancer, bladder cancer, bone cancer, brain/CNS tumors, breast cancer, leukaemia, lymphoma, melanoma cancer, osteosarcoma, ovarian cancer, rhabdomysarcoma, soft tissue sarcoma, testicular cancer, thyroid cancer, neuroblastoma, wilms tumor, non Hodkin lymphoma, Hodgkin disease, retinoblastoma, cervical cancer, colon/rectum cancer, esophagus cancer, eye cancer, gallbladder cancer, kidney cancer, laryngeal and hypopharyngeal cancer, liver cancer, lung cancer, lung carcinoid, lymphoma, malignant mesothelioma cancer, multiple myeloma cancer, nasal cancer, cavity and paranasal sinus cancer, nasopharyngeal cancer, oral cavity and oropharyngeal cancer, osteosarcoma, ovarian cancer, pancreatic cancer, penile cancer, pituitary tumors, prostate cancer, retinoblastoma, salivary gland, sarcoma, skin cancer, skin cancer melanoma, skin cancer merkel cell, small intestine cancer, stomach cancer, testicular cancer, thymus thyroid cancer, uterine sarcoma, vaginal cancer, vulgar cancer, and waldenstrom macroglobulinemia.

Cancer treatment as used herein may be surgery, chemotherapy, hormonal therapy, immunotherapy and/or radiation therapy.

Examples of chemotherapeutic agents are: doxorubicin/adriamycin, epirubicin/ellence taxanes such as paclitaxel/taxol and docetaxel/taxotere. Platinum agents such as cisplatin (Platinol, Platinol-AQ) and carboplatin ((Paraplatin), vinorelbine (Navelbine), capecitabine (Xeloda), liposomal doxorubicin (Doxil), gemcitabine (Gemzar), mitoxantrone, ixabepilone (Ixempra®), albumin-bound paclitaxel (Abraxane®), eribulin (Halaven®), cyclophosphamide (Cytoxan, Ceosar), foxorubicin (Adriamycin), etoposide (VePesid), fluorouracil (5-FU), irinotecan (Camptosar), methotrexate (Folex, Mexate, Amethopterin), paclitax topotecan (Hycamtin), Taxol, Oncovin, Vincasar PFS, and vinblastine (Velban). In an aspect of the invention the cancer treatment is chemotherapy, preferably methotrexate.

Examples of hormonal drugs for hormone therapy are LHRH (luteinizing hormone-releasing hormone) agonists such as goserelin, leuprorelin, and triptorelin and LHRH antagonist such as degarelix. Further examples are steroidal anti-androgens such as cyproterone acetate and non-steroidal anti-androgener such as bicalutamide, nilutamide, and flutamide.

The term “combination” as used herein means that the specific active ingredients are administered together in such an amount as to produce a therapeutic effect.

### Application Forms

The compositions as described above can be used as a nutritional therapy, nutritional support, as a medical food, as a food for special medical purposes or as a nutritional supplement. The compositions as described above can alternatively be used as complete nutrition as a medical food, as a food for special medical purposes or as tube feed. The nutritional compositions are advantageously provided as “ready-to-use” product, i.e. in the final form of the nutritional composition as administered to a patient that undergoes cancer treatment. Typically, the nutritional compositions herein are pre-packed in a ready to use format, i.e. sold in separately packed dose units that do not require any further dilution or reconstitution etc.,

Alternatively the nutritional composition may be provided in the form of a “supplement” or “dietary supplement” that provides nutrients to an individual that may otherwise not conveniently be consumed in sufficient quantities by said individual and may be used to complement the nutrition of an individual. It may be in the form of tablets, capsules, pastilles or a liquid and the like, preferably in the form of a liquid.

In embodiments in the nutritional composition may include further ingredients such as flavoring agents (e.g., fruit flavors, chocolate flavor, vanilla flavor, etc), emulsifying or thickening agents (e.g., lecithin, carrageenan, cellulose gum, cellulose gel, starch, gum arabic, xanthan gum, and the like); stabilizing agents, preservatives (e.g., potassium sorbate, sorbic acid, and so forth), antioxidants (e.g., ascorbic acid, sodium ascorbate, etc.), coloring agents, vitamins, minerals, and combinations thereof.

The nutritional compositions of the invention may be administered under the supervision of a medical specialist, or may be self-administered.

Optimally, the nutritional composition of the invention is consumed for the duration of patient care and cancer treatment. E.g. until the side-effect of the last cancer treatment disappeared. Since these nutritional compositions are safe to consume patients can continue taking these nutritional compositions for as long as required.

### EXAMPLES

For a more complete understanding of the present disclosure, reference is now made to the following examples taken in conjunction with the accompanying figures.

### Example 1-In Vivo Effect of a Nutritional Composition Comprising Hydrolysed Whey Protein and MCT on Tumor Growth

Tumour-bearing studies were conducted in accordance with ethical guidelines approved by the University of Adelaide Animal Ethics committee in accordance with the National Health and Medical Research Committee's Code for the care and use of animals for scientific research. All experiments were performed with N=8 rats per group.

The rats were individually-housed in conventional, open top cages under 12 h light/dark cycles with ad libitum access to autoclaved food and sterile water. Sawdust bedding was provided in all cases with enrichment (crinkle paper for tumour-naïve model or toilet rolls for tumour-bearing model). All cages were randomly arranged across racks to prevent potential bias and dampen variation.

The Dark Agouti Mammary Adenocarcinoma (DAMA) model was used to assess the effect of a composition comprising hydrolysed whey proteins and MCTs on tumor growth. Following acclimatisation (1 week with AIN93G control diet), female DA rats weighing 150-170 g were given ad libitum access to the test or control diet (table 1) for two weeks prior to methotrexate (MTX) treatment (day-14). The molecular weight distribution of the whey protein hydrolysate of the test diet is shown in table 2. On day-5 they were subcutaneously inoculated with 0.2 ml (2.0×10⁷ cells/ml) mammary adenocarcinoma cells on each flank. MTX (2 mg/kg) or vehicle control (saline) were administered intramuscularly in two separate doses 24 hours apart and rats were terminated four days after the first dose. Tumours were measured daily using digital calipers to determine their volume (cm³). Tumour burden was calculated as tumour length width*depth (volume) and expressed as relative to body weight (% BW, cm³/g). Rats were terminated 4 days after the second

TABLE 1
Dietary composition of the control and
test diet. Diets are isocaloric.

| Ingredients (g/kg) | Control | Test |
|---|---|---|
| Carbohydrates | | |
| Cornstarch | 393.40 | 250.00 |
| Dextrinized cornstarch | 132.70 | 104.50 |
| Sucrose | 100.50 | 100.00 |
| Fiber | | |
| Cellulose | 47.8 | 40.00 |
| Protein | | |
| Casein | 201.1 | 0.00 |
| Hydrolysed Whey having | 0.00 | 232.1 |
| a DH of 20% | | |
| Fat | | |
| MCT | 0.00 | 47.30 |
| Soybean oil | 70.36 | 14.60 |
| Other | | |
| Mineral Mix | 35.00 | 35.00 |
| Vitamin Mix | 10.00 | 10.00 |
| Choline bitartrate | 2.50 | 2.50 |
| tBHQ anti-oxidant | 0.014 | 0.014 |
| Total Nutritonal value | | |
| Total carbohydrates | 626.60 | 614.40 |
| Total intact protein | 201.30 | 235.7 |
| Total fat | 70.40 | 61.90 |
| Other specifications | | |
| Kcal | 3821 | 3185 |
| Total weight (g) | 1000 | 993 |
| Protein energy % (EN %) | 22.40 | 22.80 |

MTX dose via general isoflurane anaesthesia, cardiac puncture and cervical dislocation.

TABLE 2
Molecular weight distribution in kDa of the hydrolysed
whey protein fraction used in the test diet.

| Molecular weight | percent of whey protein |
|---|---|
| (Da) | hydrolysate (%) |
| <0.375 | 16 |
| 0.375-750 | 31 |
| 750-1250 | 21 |
| 1250-2500 | 24 |
| >2500 | 8 |

All rats consumed the test and control diet at the same rate in the two weeks prior to MTX treatment (Control: 20.67±1.17 g/day; MTX±Control diet (C.Diet): 19.44±1.49 g/day; MTX±Test diet (T.Diet): 20.93±1.57 g/day). Body weights were comparable throughout this time period, with differences only observed after MTX. After MTX treatment, MTX±C.Diet rats lost a significant amount of weight compared to controls and MTX±T.Diet. The test diet was also found to promote alimentation, with total food intake in the MTX±T.Diet being increased compared to the MTX±C.Diet group and promote hydration (data not shown). Mass spectrometry confirmed there was no difference in systemic MTX concentrations across treatment groups (C.Diet: 24.5±1.5 g/ml, T.Diet: 22.0±0.00 μg/ml).

Dietary intervention did not influence normal tumour growth (in the absence of MTX) in the tumour-bearing DAMA model. The tumour burden over the duration of the experiment wherein the tumour burden was calculated as tumour length*width*depth (volume) and expressed as relative to body weight (% BW, cm³/g). It is apparent that in the rats that did not receive MTX the tumour burden increases over time with the tumour burden showing a trend to be lower in the animals in the vehicle plus Test diet as compared to the animals in the Vehicle plus Control diet (data not shown).

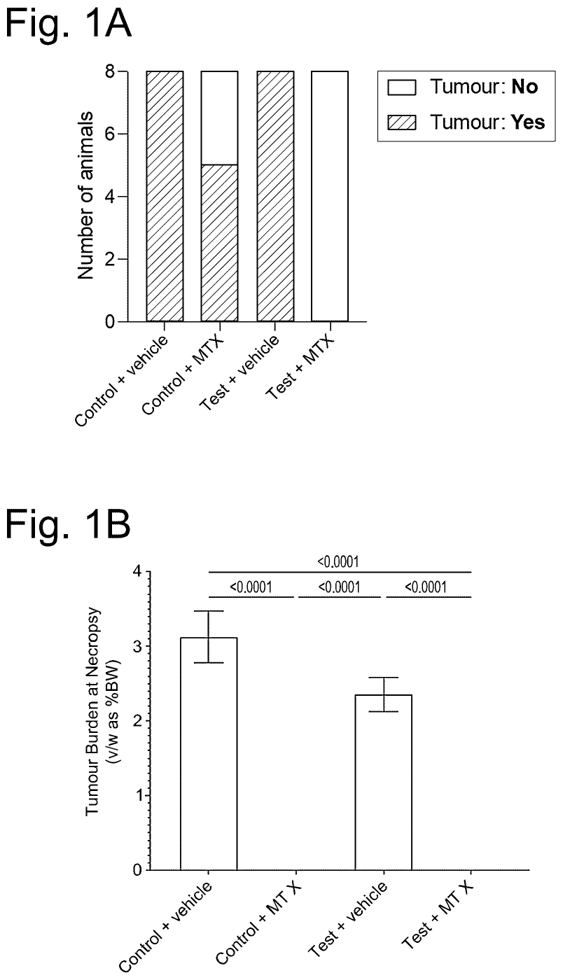

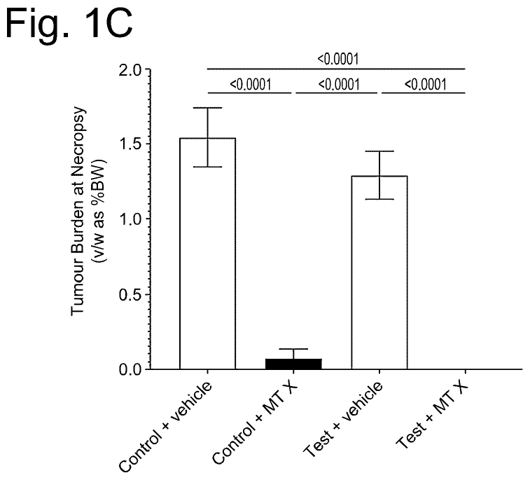

At necropsy, tumours were dissected and weighed. The number of animals having tumours after MTX treatment was significantly higher (P<0.0001, Chi squared test) in the rats fed the Control diet (5/8) than in the rats fed the Test diet (0/8) (FIG. 1A). No difference in the presence of detectable tumours was observed between the animal that were not treated with MTX and fed the Control or Test diet respectively.

The tumour burden in all experimental groups was assessed after necropsy both as relative burden of tumor volume as percentage of the bodyweight of the animals (FIG. 1B) and tumor weight relative to the bodyweight of the animals (FIG. 1C). It is apparent from FIGS. 1B and 1C that in the MTX treated rats the tumor burden is significantly decreased to (near) zero (one-way ANOVA with Tukey's post-hoc test, p<0.0001). FIG. 1C illustrates that the Test diet in MTX treated rats resulted in a further reduction of the tumor burden compared to the MTX treated rats fed the Control diet. A similar trend is observed in the animals that were not treated with MTX (vehicle) and fed the Test diet; the tumor borden is lower in rats fed the Test diet compared to rats fed the Control diet (FIGS. 1B and 1C).

Overall it was shown that systemic MTX levels were unchanged by the test diet. Furthermore, using the tumor-bearing model, it was demonstrated that the extensively hydrolysed whey protein+MCT enriched Test diet enhanced the efficacy of MTX, with 100% of the MTX-treated rats on the Test diet having full tumor clearance, compared to 62.5% of the control diet group.

### Example 2

By way of example, and not limitation, an example of a suitable composition that may be used is as follows.

An exemplary nutritional composition includes the following ingredients: water; cornstarch, medium-chain triglyceride oil, soybean oil; whey protein hydrolysate. The composition has the following nutrient composition (per 150 kcal): 18.8 g cornstarch, 7.5 g hydrolysed whey having a degree of hydrolysation of 20%, 3 g MCT oil, 2 g soybean oil. The nutritional composition has 20 EN % protein, 50.1 EN % carbohydrates, 30 EN % fat of which 18 EN % of MCT.

1-17. (canceled)

18. A method for (i) reducing tumor growth and/or (ii) improving treatment efficacy in subjects having cancer, comprising administering to the subject a nutritional composition comprising therapeutically effective amounts of (a) hydrolysed whey protein and (b) MCT, wherein the hydrolysed whey protein comprises more than 40 wt. % of whey peptide fragments having a molecular weight of between 0.5 and 3 kDa based on total weight of the hydrolysed whey protein.

19. The method according to claim 18, wherein the hydrolysed whey protein comprises more than 10 wt. % of whey proteins having a molecular weight below 750 Da based on total weight of the hydrolysed whey protein.

20. The method according to claim 18, wherein the hydrolysed whey protein comprises less than 5 wt % of whey protein fragments having a molecular weight of 5 kDa or more based on total weight of the hydrolysed whey protein.

21. The method according to claim 18, wherein the nutritional composition comprises (I) a protein fraction comprising at least 80 wt % hydrolysed whey protein based on the total weight of the protein fraction and (II) a lipid fraction comprising at least 40 wt % MCTs based on total weight of the lipid fraction.

22. The method according to claim 21, wherein the (I) protein fraction comprises at least 85 wt. % hydrolysed whey protein based on total weight of the protein fraction.

23. The method according to claim 21, wherein the MCTs provide 9-25% of the total energy content of the nutritional composition.

24. The method according to claim 21, wherein the protein fraction provides 5 to 45% of the total energy content of the nutritional composition.

25. The method according to claim 21, wherein the nutritional composition comprises a (III) carbohydrate fraction.

26. The method according to claim 21, wherein the carbohydrate fraction provides 30-70% of the total energy content of the nutritional composition.

27. The method according to claim 18, wherein (i) reducing tumor growth and/or (ii) improving treatment efficacy in a subject having cancer and undergoing cancer treatment involves
reducing tumor growth,
reducing cancer cell mitosis,
increasing cancer cell apoptosis,
improving cancer cell sensitivity to cancer treatment and/or
reducing the amount and/or occurrence of metastasis compared to the reduction in tumor growth and/or improvement of treatment efficacy in a subject having cancer and undergoing cancer treatment without use of the nutritional composition.

28. The method according to claim 18, wherein cancer treatment is selected from surgical treatment, chemotherapy, radiation therapy, immunotherapy, or combinations thereof.

29. The method according to claim 18, wherein the cancer treatment is at least chemotherapy.

30. The method according to claim 18, wherein the nutritional composition is in the form of a liquid.

31. The method according to claim 18, wherein the whey protein hydrolysate has a degree of hydrolysis of about 15 to 25%.

32. The method according to claim 18, wherein the subject is undergoing cancer treatment.
